(19) United States
(12) Patent Application Publication
(10) Pub. No.: US 2026/0216270 A1
(43) Pub. Date: Jul. 30, 2026

(54) PLANT HYDROETHANOLIC EXTRACTS AND USES THEREOF

(71) Applicant: IMMUNE & GENETICS PROTOCOLS LLC, Ocala, FL (US)

(72) Inventors: William Denizard FLORES, Ocala, FL (US); Daniel Tommy KEREKES, Montevideo (UY)

(21) Appl. No.: 19/143,307

(22) PCT Filed: Dec. 26, 2023
(86) PCT No.: PCT/IB2023/063243

§ 371 Date: Jun. 25, 2025

Related U.S. Application Data
(60) Provisional application No. 63/435,690, filed on Dec. 28, 2022.

(51) Int. Cl. A61K 36/28; A61K 36/68; A61P 25/28

(52) U.S. Cl. CPC A61K 36/28; A61K 36/68; A61P 25/28; A61K 2236/333

(57) ABSTRACT

Provided herein plant extracts useful in management of neurological and/or psychiatric disorders.

### FIELD OF THE INVENTION

The present disclosure relates to hydroethanolic plant extracts, in particular to their therapeutic use in the treatment and/or prevention of neurological or psychiatric conditions. Furthermore, the present disclosure relates to hydroethanolic extracts of the plant Vernonanthura nudilora, preferably in combination with Plantago major, and/or a Baccharis family species selected from the group consisting of Baccharis crispa, Baccharis trimera, and Baccharis articulata.

### BACKGROUND OF THE INVENTION

Natural products, such as plant remedies, are commonly used worldwide, due to their relative availability, price, laxed regulation due to traditional use, and the perceived advantages over the modern “chemical” remedies. Several natural products and their derivatives are currently clinically used, for example, natural products with anti-cancer activity such as the plant-derived paclitaxel and docetaxel. Many thousands of medicinal plants and minerals are known in Eastern and Western traditional pharmacognosy and medicine. Of these vast plurality of species, three particular plants are the subject of the present application.

Vernonanthura nudilora is a species of perennial plants in the family Asteraceae that includes more than 23,500 species spread over about 1600 genera (Vernonanthura nudilora (Less.) H. Rob.|Plants of the World Online|Kew Science. Plants of the World Online. Retrieved 2020-08-10) with distribution in Argentina, Brazil and Uruguay (Vega A J, Dematteis M. 2011. Pollen morphology of some species of Vernonanthura (Asteraceae, Vernonieae) from southern South America. Palynology. 35(1):94-102. 2011). The Vernonanthura (Vernonia) genera includes more than 1000 species (Bremer, K. (1994) Asteraceae: Cladistics and Classification. Timber Press, Portland). The anti-proliferative and antioxidant activities of an organic extract of Vernonanthura nudilora and of some of the chemical constituents thereof was reported by Ramos, A. V. G. et al. 2019 (Ramos, A. V. G. et al., J. Braz. Chem. Soc. 2019, Vol. 30 (8), 1728-1740). In addition, some metabolites isolated from the flowers of Vernonanthura nudilora showed antimicrobial activities (A. V. G. Ramos et al., 2021, The chemistry of Vernonanthura nudiflora (Less.) H. Rob. flowers and its antimicrobial activities, Natural Product Research, doi: 10.1080/14786419.2021.1980790).

Plants of Baccharis family have found significant use in traditional South-American medicine. Among them Baccharis trimera (B. trimera) is the most studied one, and has been reported in traditional treatment of gastrointestinal disorders and hepatic diseases (Garcia et al. 2014 A comparative study of two clerodane diterpenes from Baccharis trimera (Less.) DC on the influx and mobilization of intracellular calcium in rat cardiomyocytes. Phytomedicine 21:1021-1025; Losqui Y R, Rozete F S S, Almeida M B et al (2009) Activity of Baccharis trimera (Less.) DC. Asteraceae on culture of retinal ganglion cells in vitro. Rev Bras Fannacogn 19:931-936) and several biological activities have been reported for this plant, including antihepatotoxic, antidiabetic, schistosomicidal, antioxidant, antinociceptive, and anti-inflammatory effects that are attributable to flavonoids, diterpenes, triterpenes, saponins, essential oils, and caffeoylquinic acids (Abad M J, Bermejo P (2007) Baccharis (Compositae): a review update. Arkivoc 7:76-96; Campos F R, Bressan J, Jasinski V C et al (2016) Baccharis (Asteraceae): chemical constituents and biological activities. Chem Biodivers 13: 1-17). A hydroethanolic extract of B. trimera was also reported (Francislaine Aparecida Dos Reis Livero, Luisa Mota da Silva et al., Hydroethanolic extract of Baccharis trimera promotes gastroprotection and healing of acute and chronic gastric ulcers induced by ethanol and acetic acid Naunyn Schmiedebergs Arch Pharmacol. 2016 September; 389(9):985-98). Several Baccharis species are used interchangeably, e.g., B. trimera, B. crispa, and B. articulata, with the two former being even very difficult to distinguish between.

Furthermore, the plant Plantago major (P. major) commonly known as “great plantain”, is a widespread used medicinal plant from the Plantaginaceae family (Wang H, Zhao C, Huang Y, Wang F, Li Y, Chung H Y. Chemical Constituents and Bioactivities of Plantaginis Herba. Hong Kong Med J. 2015; 22:29-35). Plantago major contains several active compounds, such as flavonoids, polysaccharides, terpenoids, lipids, iridoid glycosides and caffeic acid derivatives (Samuelsen A B. The traditional uses, chemical constituents and biological activities of Plantago major L. A review. J Ethnophannacol. 2000; 71:1-21) and is used in treatment of various diseases such as constipation, coughs, wounds, infection, fever, bleeding and inflammation (Aghili M. Makhzan-O-L Advieh. Tehran: Tehran University of Medical Science Press; 2008. (3)). In addition, water and ethanol extracts of Plantago major leaves show anti-inflammatory activity on oral epithelial cells (Zubair et al. Journal of Traditional and Complementary Medicine 9 (2019) 169e171170).

There remains an unmet need in the art to identify and harness the natural products, such as the ones produced by V. nudilora, B. crispa/articulata/itrmera, and/or P. major, for the use, inter alia, in treating neurological and/or psychiatric conditions.

### SUMMARY OF THE INVENTION

It has now been unexpectedly found that extracts of the plant V. nudilora, in particularly combination with extracts of B. crispa/articulata/trimera, and/or P. major, can be used in the treatment of various neurological and/or psychiatric diseases or disorders, e.g., neurodevelopmental or neuroinflammaroty, e.g., developmental disorders such as development retardation or dementia associated with trisomy 21 (Down Syndrome), autism spectrum disorder (ASD, for example, Asperger's Syndrome), mental health conditions, such as anxiety disorder and schizophrenia, and other neurological disorders, for example alternating hemiplegia of childhood, epilepsy, neurodegenerative disorders, and others. Without being bound to a particular narrative it is believed that several of the compounds present in these herbal extracts have neuro-protective, pro-survival, and/or anti-inflammatory effects, and are responsible for the beneficial properties of the extracts as demonstrated in the present disclosure.

Therefore, in a first aspect thereof the present disclosure provides a method of treating a neurological and/or psychiatric disorder in a subject in need thereof, comprising administering to said subject a composition comprising a plant-derived material, e.g., a hydroethanolic plant extract or any fraction thereof, wherein said plant is selected from Vernonanthura nudilora, Plantago major, and a Baccharis family species selected from Baccharis crispa, Baccharis trimera, and Baccharis articulata, and any combination thereof. For example, the Baccharis family species is Baccharis articulata. The extract is preferably obtainable by a process, e.g., essentially identical to an extract obtained by the process, wherein the process comprises the steps of combining the biomass of said plant(s) with a hydroethanolic mixture comprising 20 to 50 volume percent of water, and extracting said biomass into said hydroethanolic mixture for a time interval of between about 3 and 35 days, e.g., between about 10 and 30 days, preferably between about 18 and 23 days. Preferably the concentration of the biomass being extracted is between 13% and 33% weight by volume of the total extraction mixture, preferably about 15% and 25%, e.g., between 18% and 22% weight by volume of the total extraction mixture. The extracting may be carried out in a ratio of between 1:2 to 1:8, e.g., between 1:3 and 1:6 between the biomass being extracted and the hydroethanolic mixture, preferably about 1:4. Preferably, the extracting is conducted at a temperature between about 15° C. and 40° C., further preferably, between 20° C. and 25° C.

In currently preferred embodiments, the method as disclosed herein is wherein the plant is a combination of Vernonanthura nudiflora, Plantago major, and a Baccharis family species selected from Baccharis crispa, Baccharis trimera, and Baccharis articulata, preferably wherein a weight ratio of each of the plants is between 20 and 50% wt, e.g., such that the ratio between the plants is 2:2:1 with Plantago major being the minor component. In some preferred embodiments, the extract contains less than 5-20% by weight of the compounds listed in Table 1 hereinbelow, or is essentially devoid of these compounds.

The method as described herein is wherein the neurological and/or psychiatric disorder is a disease or disorder is classified in a subclass 06 or 08 of the 11^{th} revision of World Health Organization International Classification of Diseases, e.g., wherein said neurological and/or psychiatric disorder is a neurodevelopmental disorder, an anxiety or fear-related disorder, a primary psychotic disorder, or a seizures disorder. In currently preferred embodiments, the developmental disorder may be an autism spectrum disorder, e.g., Asperger's syndrome, an attention deficit hyperactivity disorder, or Down Syndrome-associated development retardation. The anxiety or fear-related disorder may be a primary psychotic disorder, e.g., schizophrenia. The seizures disorder may be an epilepsy, or a hemiplegia.

In some embodiments, the method as described herein is wherein the hydroethanolic plant extract is a dried hydroethanolic plant extract, but may also be wherein said extract is a liquid hydroethanolic plant extract.

In some embodiments as described herein, the method may comprise administering to a subject in need thereof a therapeutically effective amount of the composition. Optionally, the administering may be by a route selected from the group consisting of intravenous administration, oral administration, intramuscular administration, and intraperitoneal administration. Optionally, the method as disclosed herein may be wherein the administering comprises providing to the patient a daily amount of plant extract equivalent to a dose between 10 mg and 300 mg, on dried basis, divided into doses according toa suitable regimen. Dosages (i.e., therapeutically effective amounts) may vary between 0.008 mL/kg (kg-weight of the patient) and 2 mL/kg of the diluted 1:8 hydroethanolic extracts, i.e., between 0.001 mL/kg and 0.25 mL/kg of concentrated mL, which means between 0.033 mg/kg (of dry mass/kg of patient) and 8.6 mg/kg (of diy mass/kg of patient). Preferably, dosages (i.e., therapeutically effective amounts) may vary between 0.048 mL/kg (kg-weight of the patient) and 1 mL/kg of the diluted 1:8 hydroethanolic extracts, i.e., between 0.006 mL/kg and 0.125 mL/kg of concentrated mL, which means between 0.2 mg/kg (of dry mass/kg of patient) and 4.3 mg/kg (of dry mass/kg of patient). The dose is preferably expressed based on the dry basis of the extract. Thus, in some embodiments, the dose may vary between 25 and 500 mg per day. Optionally, the dose may be between 10 mg and 120 mg on dry basis, preferably wherein said neurological and/or psychiatric disorder is a neurodevelopmental disorder; or optionally the dose may be between 100 mg and 300 mg on dry basis, preferably wherein said neurological and/or psychiatric disorder is a primary psychotic disorder. The method as disclosed herein may be wherein the regimen is selected from once daily, twice daily, three times daily, and four times daily. In some further embodiments, the method as disclosed herein may be wherein said method further comprises administering to said subject an additional therapeutic agent.

In a further aspect provided herein a composition comprising a hydroethanolic plant extract or any fraction thereof, for use in a method as generally disclosed herein. In a further aspect provided herein a dosage form comprising the composition as disclosed herein, preferably the dosage form being selected from the group consisting of a tincture, a syrup, an elixir, a capsule, and a tablet.

### DETAILED DESCRIPTION OF THE INVENTION

Vernonanthura nudilora extracts have been prepared in the past by exhaustive organic ethanolic extraction, followed by fractionation into a variety of organic solvents. Some of the compounds identified in these extracts, for example, in the abovementioned publication by Ramos A. V. G. et al., 2019, namely piptocarphin sesquiterpene lactones, in particular, 8α-tigloyloxy-10α-hydroxy-hirsutinolide, some triterpenes, glycosylated steroids, some flavonoids, including velutin, and chlorogenic acid derivatives, have been also tested for anti-cell proliferation activity in cell lines with a varying degree of success. The compounds identified in Ramos et al 2019 are presented in Table 1 below.

It has now been unexpectedly found that V. nudilora together with the plants Baccharis trimera articulata and Plantago major, when subjected to hydroethanolic extraction, as opposed to organic ethanolic extraction, for prolonged time intervals, furnishes an extract that has a beneficial therapeutic effect in a variety of neurological and/or psychiatric conditions, e.g., developmental disorders, mental health conditions and neurodegenerative conditions and/or inflammatory conditions, such as a neurodevelopmental disorder, an anxiety or fear-related disorder, a primary psychotic disorder, or a seizures disorder. As demonstrated in the appended examples, the administration of a mixture of hydroethanolic extracts of Vernonanthura nudilora, Baccharis articulata, and Plantago major, provided unexpected therapeutical benefit to patients with autism spectrum disorders, seizures, schizophrenia, and development retardation/deterioration originating from trisomy 21.

TABLE 1

| No | Name | IUPAC name | CAS No. |
|---|---|---|---|
| 1 | Lupeol | (1R,3aR,5aR,5bR,7aR,9S,11aR,11bR,13aR,13bR)-3a,5a,5b,8,8,11a- | 545-47-1 |
| | | hexamethyl-1-prop-1-en-2-yl-1,2,3,4,5,6,7,7a,9,- | |
| | | 10,11,11b,12,13,13a,13b-hexadecahydrocyclopenta[a]chrysen-9-ol | |
| 2 | α-Amyrin | (3S,4aR,6aR,6bS,8aR,11R,12S,12aR,14aR,14bR)-4,4,6a,6b,8a,- | 638-95-9 |
| | | 11,12,14b-octamethyl-2,3,4a,5,6,7,8,9,10,11,12,12a,14,14a- | |
| | | tetradecahydro-1H-picen-3-ol | |
| 3 | β -Amyrin | 3S,4aR,6aR,6bS,8aR,12aR,14aR,14bR)-4,4,6a,6b,8a,11,11,14b-octa- | 559-70-6 |
| | | methyl-1,2,3,4a,5,6,7,8,9,10,12,12a,14,14a-tetradecahydropicen-3-ol | |
| 4 | Taraxasterol | (3S,4aR,6aR,6bR,8aR,12S,12aR,12bR,14aR,14bR)- | 1059-14-9 |
| | | 4,4,6a,6b,8a-,12,14b-Heptamethyl-11-methylidenedocosahydropicen-3-ol | |
| 5 | Pseudotaraxa- | 4,4,6a,6b,8a,11,12,14b-octamethyl-2,3,4a,5,6,6a,7,8,- | 464-98-2 |
| | | 9,12,12a,13,14,14a-tetradecahydro-1H-picen-3-ol | |
| | sterol | | |
| 6 | Piptocarphin A | (4S,6R,7S,10R,11E)-5,6,7,8,9,10-Hexahydro-3-(acetoxymethyl)-6,7- | 76248-63-0 |
| | | dihydroxy-4-(methacryloyloxy)-6,10-dimethyl-7,10- | |
| | | epoxycyclodeca[b]furan-2(4H)-one | |
| 7 | Piptocarphin B | (E)-3-(acetoxymethyl)-6,7-dihydroxy-6,10-dimethyl-2-oxo- | 76215-49-1 |
| | | 2,4,5,6,7,8,9,10-octahydro-7,10-epoxycyclodeca[b]furan-4-yl (E)-2- | |
| | | methylbut-2-enoate | |
| 8 | Piptocarphin D | [(2Z)-8,10,11-trihydroxy-1,10-dimethyl-5-oxo-4,14- | 76215-51-5 |
| | | dioxatricyclo[9.2.1.03,7]tetradeca-2,6-dien-6-yl]methyl acetate | |
| 9 | 8α-tigloyloxy-10αhydroxy-hirsutinolide: (E)-3-ethyl-6,7-dihydroxy-6,10-dimethyl-2-oxo-2,4,5,6,7,8,9,- | | |
| | 10-octahydro-7,10-epoxycyclodeca[b]furan-4-yl (E)-2-methylbut-2-enoate | | |
| 10 | Velutin | 5-Hydroxy-2-(4-hydroxy-3-methoxyphenyl)-7-methoxy-4H-1- | 25739-41-7 |
| | | benzopyran-4-one | |
| 11 | Apigenin | 5,7-Dihydroxy-2-(4-hydroxyphenyl)-4H-1-benzopyran-4-one | 520-36-5 |
| 12 | Chrysoeriol | 5,7-Dihydroxy-2-(4-hydroxy-3-methoxyphenyl)-4H-1-benzopyran- | 491-71-4 |
| | | 4-one | |
| 13 | β-Sitosterol β-D-glucoside (sitosterol-3-β-O-D-glicopyranoside) | | 474-58-8 |
| 14 | Stigmasterol-3-β-O-D-glicopyranoside | | |
| 15 | 3-O-caffeoyl- | (1S,3R,4R,5R)-3-{[(2E)-3-(3,4-Dihydroxyphenyl)prop-2- | 202650-88-2 |
| | quinic acid | enoyl]oxy}-1,4,5-trihydroxycyclohexane-1-carboxylic acid | |
| 16 | Rutin | (42S,43R,44S,45S,46R,72R,73R,74R,75R,76S)- | 153-18-4 |
| | | 13,14,25,27,43,44,45,73,74,75-Decahydroxy-76-methyl-24H-3,6- | |
| | | dioxa-2(2,3)-[1]benzopyrana-4(2,6),7(2)-bis(oxana)-1(1)- | |
| | | benzenaheptaphane-24-one | |
| 17 | Luteolin | 2-(3,4-Dihydroxyphenyl)-5,7-dihydroxy-4H-1-benzopyran-4-one | 491-70-3 |
| 18 | (E)-3-(acetoxymethyl)-6-hydroxy-6,10-dimethyl-2-oxo-2,4,5,6,9,10-hexahydro-7,10- | | |
| | epoxycyclodeca[b]furan-4-yl (E)-2-methylbut-2-enoate | | |
| 19 | (E)-3-(acetoxymethyl)-6-hydroxy-6,10-dimethyl-2-oxo-2,4,5,6,9,10-hexahydro-7,10- | | |
| | epoxycyclodeca[b]furan-4-yl methacrylate | | |
| 20 | 3-(acetoxymethyl)-7,11-dihydroxy-6,10-dimethyl-2-oxo-2,4,5,6,7,8,9,10,11,11a-decahydro-7,10- | | |
| | epoxycyclodeca[b]furan-4-yl methacrylate | | |
| 21 | 3-(acetoxymethyl)-7,11-dihydroxy-6,10-dimethyl-2-oxo-2,4,5,6,7,8,9,10,11,11a-decahydro-7,10- | | |
| | epoxycyclodeca[b]furan-4-yl (E)-2-methylbut-2-enoate | | |
| 22 | 8α-Tigloyloxy- | 2-Butenoic acid, 2-methyl-, (4S,6R,7S,10R,11E)-3- | 83182-58-5 |
| | hirsutinolide 13- | [(acetyloxy)methyl]-2,4,5,6,7,8,9,10-octahydro-7-hydroxy-6,10- | |
| | O-acetate | dimethyl-2-oxo-7,10-epoxycyclodeca[b]furan-4-yl ester, (2E)- | |
| 23 | Piptocarphin E | [(2E)-6-(acetyloxymethyl)-11-ethoxy-10-hydroxy-1,10-dimethyl-5- | 76215-52-6 |
| | | oxo-4,14-dioxatricyclo[9.2.1.03,7]tetradeca-2,6-dien-8-yl] 2- | |
| | | methylprop-2-enoate | |
| 24 | Diacetyl | (E)-(4-acetoxy-6,7-dihydroxy-6,10-dimethyl-2-oxo-2,4,5,6,7,8,9,10-octahydro-7,10- | |
| | piptocarphol | epoxycyclodeca[b]furan-3-yl)methyl acetate | |
| 25 | 5-acetoxy-8-(acetoxymethyl)-1a,5-dimethyl-4,9-dioxo-1a,2,3,4,5,6,7,9,10a,10b- | | |
| | decahydrooxireno [2′,3′:9,10]cyclodeca[1,2-b]furan-7-yl (E)-2-methylbut-2-enoate | | |
| 26 | Glaucolide A | 5-acetoxy-8-(acetoxymethyl)-1a,5-dimethyl-4,9-dioxo- | 11091-29-5 |
| | | 1a,2,3,4,5,6,7,9,10a,10b-decahydrooxireno[2′,3′:9,10]cyclodeca[1,2- | |
| | | b]furan-7-yl methacrylate | |

Therefore, in a first aspect thereof the present disclosure provides a method of treating a neurological and/or psychiatric disorder in a subject in need thereof, comprising administering to a patient in need thereof a composition comprising a plant-derived material, e.g., a hydroethanolic plant extract or any fraction thereof, wherein said plant is selected from Vernonanthura nudilora, Plantago major and a Baccharis family species selected from Baccharis crispa, Baccharis trimera, and Baccharis articulata, and any combination thereof. In particular embodiments, the Baccharis family species suitable for the methods of the preset disclosure is Baccharis articulata. In further embodiments, the Baccharis family species suitable for the methods of the preset disclosure is Baccharis trimera.

It has now been unexpectedly found that prolonged hydroethanolic extraction of plant substance, as described hereinbelow, provides an extract with beneficial effect for treating subjects afflicted, inter alia, with neurological and/or neurodevelopmental disorders. The extract as herein defined is obtainable by a process as described in greater detail herein. That is, the extract may be prepared by the process, or may be prepared by a different process, provided that the obtained extract is essentially identical to an extract obtained by the process as described herein. The essentially identical extract may have at least 60% of the similarity in the composition with the extract prepared by the process as described herein. Preferably, the essentially identical extract has at least 65% similarity, or at least 70% similarity, or 75, 80, 85, 90, or at least 95% similarity. The similarity may be conventionally determined as known in the art, e.g., by comparing output of analytical methods, such as mass-spectrometry coupled chromatography, e.g., LC-MS/MS, GC-MS/MS. The peak positions, the molecular weight of the compounds, and/or their fragmentation patterns may be used to assess the similarity. Additionally, suitable statistic and/or chemometric methodology may be employed to determine the similarity and the extent thereof. In a specific embodiment, the extract essentially identical to an extract obtained by the process as described herein may be characterized by the compounds present in Tables 2-9, enumerating various compounds found in the hydroethanolic extracts of Vernonanthura nudiflora, Baccharis articulata/trimera, and Plantago major.

Thus, the extract may be essentially identical to an extract prepared by an extracting process comprising combining the biomass of at least one of V. nudiflora, B. atriculata/trimera, and P. major with a hydroethanolic mixture comprising between 20 and 50 volume percent of water in ethanol. The extraction may be carried out over time intervals of between 3 and 35 days, e.g., between 10 and 30 days, preferably between 18 and 23 days, e.g., three weeks. Preferably, the extraction of plant biomass into the hydroethanolic mixture is performed in a suitable vessel, equipped by agitation means, and is agitated intermittently, with a periodicity of between 24 and 48 hours, for intervals of between 1 and 2 hours. The extracting step may be advantageously performed in an ambient temperature, e.g., between 15 and 25° C. However, the extracting may be performed at a slightly elevated temperatures, to facilitate and/or accelerate the extraction, e.g., at a temperature of between 25 and 45° C. Therefore, the temperature may be adjusted according to the needs of the process, between 15 and 45° C., but preferably the temperature would be between 2° and 25° C. In specific embodiments, extracting of plant biomass into the hydroethanolic mixture is performed at a temperature range of between about 15° C. and 40° C., preferably between 20° C. and 25° C.

The hydroethanolic mixture comprises ethanol, in a concentration of between 40 and 80 volume percent. The balance of the hydroethanolic mixture consists essentially of water. Preferably, the concentration of ethanol is between 65 and 75 volume percent. More preferably, the concentration of ethanol is about 70%. Without being bound by a particular theory, it is also currently believed that utilizing 70% ethanol for extraction medium may be particularly advantageous due to the antiseptic properties of the solvent, thereby contributing to the microbial stability of the extracts in their liquid form.

V. nudilora biomass as well as biomass of the plants Baccharis trimera articulata and Plantago major (namely plant-based material) may be obtained by harvesting the plants, checking the quality of the aerial parts of the plants, i.e., parts residing above the soil, including the stems, leaves, petioles, flowers, fruit, and seeds, cleaning and/or washing the plant material to remove loose dirt and contaminants, and drying the clean aerial plant parts. In particular, stems, leaves, petioles and seeds aerial parts of the plants may be useful for preparing the extract of the present disclosure. In particular embodiments, the process comprises extracting the aerial parts of said plant(s). In some embodiments, the extraction process as herein defined comprises drying of the plant prior to extracting. The drying is preferably carried out indoors at ambient or slightly elevated temperature, i.e., between 15 and 45° C., preferably between 2° and 25° C. The drying may also be carried out at slightly elevated temperatures, e.g., 40-50° C. The drying may usually be carried out until at least 40% weight loss of the original plant material mass is obtained. The drying may be continued until between 40 and 60 weight percent weight loss of the initial weight of the plant is obtained. Such weight loss is usually achieved after 7-8 days at room temperature and after 3-4 days at a temperature of 45° C. The dried biomass may then be ground using a suitable size-reduction equipment, such as a mill, e.g., dried biomass is size reduced using electric a hammer mill. Dried milled biomass obtained presents a particle size of 5.0-10.0×1.0-2.0×0.2-1.0 mm. Dried ground plant biomass may then be used in extracting steps of the process.

Unlike exhaustive extractions wherein the extraction mixture is exposed to high volumes, concomitantly or repeatedly in smaller portions, of extraction solvent, the extraction process according to the present disclosure relies on the same amount of extracting solvent. Therefore, the concentration of the botanical biomass can be controlled as required. Usually, the initial concentration of the plant being extracted (i.e., during the extracting step) is between about 10% and 30%, e.g., between 15% and 25% of weight by volume of the extraction solvent. Preferably, the concentration of the biomass is between 18 and 22% weight by volume, e.g., about 20%. In other words, the extracting may be carried out in a ratio of between 1:2 to 1:8, e.g., 1:3 and 1:6 between the biomass being extracted and the hydroethanolic mixture, preferably about 1:4.

After the completion of the extraction step, namely the completion of extracting the biomass into the hydroethanolic mixture, the extract may be separated from the depleted plant biomass to furnish said extract. In other words, in some embodiments the process comprises separating remnants, i.e., residues, of said plant biomass from said hydroethanolic mixture to furnish said extract. The separating of the extract may be performed by decantation of the extract from the biomass sediment. Alternatively, or additionally, the spent biomass may be separated by filtration via a suitable filter. The filtering may be performed via a suitable filter, e.g., even a cheesecloth, or may be performed through a filter with defined pore size, e.g., cellulotic 15 μm filter. Alternatively, filtration can be performed by gravity, or using electrical devices with the above pore size. In a further embodiment, the filtration may be a vacuum filtration. The separating may also comprise, additionally or alternatively, centrifugation of the mixture to effect complete sedimentation of the spent biomass and to enable easy decantation of the extract.

The extract of the present disclosure may be a dried hydroethanolic extract, namely a plant-derived solid content (dried plant-derived content after evaporating the solvent) or the extract may be a liquid hydroethanolic extract. The drying of the primary hydroethanolic extract may be performed as known in the art, e.g., by lyophilization. If lyophilization is performed, the liquid hydroethanolic extract may be subjected to evaporation of ethanol, e.g., under vacuum, or at elevated temperature. The concentrated hydroethanolic extract comprising little amounts or but traces of ethanol, containing essentially water as the liquid carrier, may then be mixed with a suitable carrier, and then lyophilized. In one particular embodiment, the carrier may be a starch.

In some embodiments, the resulting extract as herein defined has a plant-derived solid content (dried plant-derived content after evaporating the solvent, interchangeably referred to herein in terms “dry basis”, “dried extract”, and the like) at a weight of between 0.5% and 15.0%, preferably between 1.0% and 5.0%, or preferably between 1.8% and 4.5% weight by volume of the extract's initial volume. Further preferably, the dried basis of the extract is between 3 and 4 percent w/v in the hydroethanolic extract. In some cases, it may be advantageous to dilute the extract, either as a solid or as a dissolved solid, with further solvent or other diluting medium (e.g., similar or identical to the extraction hydroethanolic mixture, that is the diluted extract may be diluted with a hydroethanolic solution). In various embodiments, the original extract (i.e., comprising dissolved solid) may be further diluted, e.g., by dilution of 1 part of extract with 9 parts of distilled water or 2 parts of extract with 8 parts of distilled water. Another suitable dilution is 1 part of extract to 8 parts of distilled water.

It is evident that the hydroethanolic extract, e.g., of Vernonantura nudilora, is substantially different from the extract demonstrated in Ramos et al 2019. Therefore, the hydroethanolic extract as described herein preferably contains less than 20% by weight of the compounds enumerated in Table 1. Preferably, the extract of a fraction thereof comprise less than 5% by weight of the compounds enumerated in Table 1, or is essentially devoid of these compounds, e.g., includes between 0 and up to 4% by weight of the above compounds, up to 3%, to 2%, or includes up to 1% by weight of the above compounds.

Conversely, the hydroethanolic extract as described herein preferably contains more than 60%, as elaborated above, of components enumerated in tables 2-9 (showing the composition of Vernonanthura nudilora, Baccharis trimera and Plantago major).

Additionally, the hydroethanolic extract as herein defined may be further subjected to at least one further sub-extraction steps by an organic solvent, e.g., by at least one of hexane, ethyl acetate, chloroform, and butanol. In this case the hydroethanolic extract may be interchangeably referred to as “fraction” or hydroethanolic extract, unless the context clearly prescribes otherwise. It is evident that the process furnishes a liquid hydroethanolic plant extract. However, as detailed above, the extract may be dried using suitable drying means, e.g., under reduced pressure or lyophilized (for example as exemplified below), to furnish dried hydroethanolic extract. Dried ethanolic extract may further be adsorbed onto a suitable inert carrier, to be used as a powder. Alternatively, or additionally, the liquid hydroethanolic extract may be dried in presence of at least a portion of the inert carrier, to furnish adsorbed dried hydroethanolic extract.

TABLE 2

| Vernonantura plant extract | Baccharis plant extract | Plantago plant extract |
|---|---|---|
| 1) Hexadecanoic acid ethyl ester | 1) Hexadecanoic acid, ethyl ester | 1) Hexadecanoic acid, ethyl ester |
| (Peak 1) Area (Ab*s) = 33,110,310 | (Peak 26) Area (Ab*s) = 10,220,250 | (Peak 7) Area (Ab*s) = |
| Quality = 99 | Quality = 99 | 50,748,144 Quality = 99 |
| 2) Phytol (Peak 2) Area | 2) Phytol (Peak 29) Area | 2) Phytol (Peak 8) Area (Ab*s) = |
| (Ab*s) = 13,850,839 Quality = 99 | (Ab*s) = 3,989,191 Quality = 93 | 18,787,619 Quality = 96 |
| 3) Linoleic acid ethyl ester (Peak | 3) Linoleic acid ethyl ester 9,12- | 3) Linoleic acid ethyl ester (Peak |
| 3) \|ETHYL (9z,12z)-9,12- | Other names: Octadecadienoic | 11) Area (Ab*s) = 30,507,168 |
| Octadecadienoate | acid, ethyl ester (Peak 30) Area | Quality = 99 |
| (Ab*s) = 20,555,645 Quality = 99 | (Ab*s) = 5,514,452 Quality = 99 | |
| 4) 9,12,15-Octadecatrienoic acid | 4) 9,12,15-Octadecatrienoic acid, | 4) 9,12,15-Octadecatrienoic acid, |
| ethyl ester, (Z,Z,Z)- (Peak 4) | ethyl ester, (Z,Z,Z)- | ethyl ester, (Z,Z,Z)- (Peak 12) |
| (Ab*s) = 23,074,287 Quality = 99 | (Peak 31) Area (Ab*s) = 5,710,334 | Area (Ab*s) = 63,412,281 |
| | Quality = 99 | Quality = 99 |
| 5) Octadecanoic acid ethyl ester | 5) Benzene, 1-methoxy-2- | 5) Octadecanoic acid, ethyl ester |
| (Peak 5) (Ab*s) = 3,107,973 | (1-methylethenyl)- | (Peak 13) Area (Ab*s) = |
| Quality = 99 | | 4,630,724, Quality = 99 |
| 6) 1,2-Benzenedicarboxylic acid, | 6) 4H-Pyran-4-one,2,3-dihydro- | 6) 4H-Pyran-4-one,2,3- |
| bit (2-ethylhexyl) ester (Peak 6) | 3,5-dihydroxy-6-methyl- (Peak | dihydro-3,5-dihydroxy-6- |
| | 3)) Area (Ab*s) = 24,981,669 | methyl- (Peak 2)) Area |
| | Quality = 96 | (Ab*s) = 27,382,476 |
| | | Quality = 95 |
| 7) 2,5 Dimethyldiphenylsulfone | 7) 2-Methoxy-4-vinylphenol | 7) 9,12,15-Octadecatrienoic |
| | (Peak 10) | acid, (Z,Z,Z)- (Peak 10) Area |
| | Area (Ab*s) = 7,305,730 | (Ab*s) = 47,918,691 |
| | Quality = 97 | Quality = 99 |
| 8) Stigmasta-5,22-dien-3-ol (Peak | 8) 4-Methyleneisophorone | 8) 2-Methoxy-4-vinylphenol |
| 11) Area (Ab*s) = 11,567,27423. | (peak 6) Area (Ab*s) = | (Peak 3)) Area (Ab*s) = |
| | 21,458,921 Quality = 97 | 3,985,200 Quality = 94 |
| 9)OLEAN-12-EN-3-OL(Peak 8) | 9)1H-Cycloprop[e]azulen-7-ol, | 9) 9,12-Octadecadienoic acid |
| (Ab*s) = 143,738,307 | decahydro-1,1,7-trimethyl-4- | (Z,Z)- (Peak 9) Area |
| Quality = 93 | methylene-, [1ar(1a.alpha., | (Ab*s) = 8,205,041 |
| | 4a.alpha.,7.beta.,7a.beta.,7b.alpha.)]- | Quality = 99 |
| | (Peak 18) Area (Ab*s) = | |
| | 13,447,105 Quality = 99 | |
| 10) URS-20(30)-EN-3-OL | 10)1,1,4,7 tetramethyldecahydro- | 10) n-Hexadecanoic acid |
| | 1h-cyclopropa[e]azulen-4-OL, | (Peak 6) Area (Ab*s) = |
| | (Peak 19) Area (Ab*s) = | 25,158,853 Quality = 93 |
| | 60,683,772 Quality = 97 | |
| 11) (3.beta.21.beta.)-A′- | 11) Ledol | 11) Hexadecanoic acid, 2- |
| neogammacer-22(29)-en-3-ol | | hydroxy-1-(hydroxymethyl) |
| (Peak 10) Area (Ab*s) = | | ethylester |
| 59,020,551 Quality = 93 | | |
| 12) Pentacyclic Triterpene - | 12) 4aH-Cycloprop[e]azulen-4a- | 12)1,3,12-Nonadecatriene |
| Alcohol (Peak 11) Area (Ab*s) = | ol, decahydro-1,1,4,7-tetramethyl-, | |
| 22,398,502 Quality = 93 | [1aR(1a.alpha.,4.beta.,4a.beta., | |
| | 7.alpha.,7a.beta.,7b.alpha.)]- | |
| | (Peak 17) Area (Ab*s) = 30,609,427, | |
| | Quality = 99 | |
| | 13) 2(10)-Pinene (Peak 2)) | 13) gamma.-Sitosterol (Peak |
| | Area (Ab*s) = 5,807,189 | 22) Area (Ab*s) = 35,635,132 |
| | Quality = 95 | |
| | 14) Trans-Sinapyl alcohol | |
| | (Peak 27) Area (Ab*s) = | |
| | 5,523,775 Quality = 96 | |
| | 15) 2-Naphthalenemethanol, | |
| | decahydro-.alpha,.alpha.,4a- | |
| | trimethyl-8-methylene-, [2R- | |
| | (2.alpha.,4a.alpha.,8a.beta.)]- | |
| | (Peak 23) Area (Ab*s) = | |
| | 26,189,433 Quality = 97 | |
| | 16) 4-oxo-adamantane-1- | |
| | carboxylic acid methyl ester | |
| | Pyrene, 1,2,3,6,7,8-hexahydro- | |
| | (Peak 28) Area (Ab*s) = | |
| | 28,617,055 Quality = 90 | |
| | 17) 2-Hydroxy-3,5,5-trimethyl- | |
| | cyclohex-2-enone, (peak 4) | |
| | Area (Ab*s) = 10,619,964 | |
| | Quality = 96 | |
| | 18) 3-Cyclohexen-1-ol,5- | |
| | methylene-6-(1-methylethenyl)- | |
| | (peak 5)) Area (Ab*s) = | |
| | 34,153,434 Quality = 91 | |
| | 19) (4H)1-Benzopyran-4-one, | |
| | 3,5,6,7-tetrahydroxy-8-methyl- | |
| | 2-phenyl- | |
| | 20) Valerena-4,7(11)-diene | |
| | (peak 15 Area (Ab*s) = | |
| | 6,007,4363 Quality = 96 | |

TABLE 3

| Compound | | | Calc. | RT | |
|---|---|---|---|---|---|
| No. | Compound Name | Formula | MW | [min] | Area A |
| A1 | Aspara gine | C4 H8 N2 O3 | 132.05 | 0.652 | 90,024,032 |
| A2 | 11-methyl-2,7- | C19 H20 O6 | 344.12 | 8.393 | 66,445,827 |
| | dimethylidene-6,12-dioxo- | | | | |
| | 5,14- | | | | |
| | dioxatricyclo [9.2.1.0â′,â,]tetradec- | | | | |
| | 1(13)-en-9-yl2- | | | | |
| | methylprop-2-enoate | | | | |
| A3 | Myristicin (4-methoxy-6- | C11 H12 O3 | 192.07 | 8.752 | 125,507,328 |
| | (prop-2-en-1-yl)-2H-1,3- | | | | |
| | benzodioxole) | | | | |
| A4 | (3aS,4S,5S,11aR)-6-formyl- | C19 H22 O7 | 362.13 | 9.978 | 604,917,241 |
| | 5-hydroxy-10- | | | | |
| | (hydroxymethyl)-3- | | | | |
| | methylidene-2-oxo- | | | | |
| | 2H,3H,3aH,4H,5H,8H,9H,11aH- | | | | |
| | cyclodeca[b]furan-4-yl 2- | | | | |
| | methylprop-2-enoate | | | | |
| A5 | Calealactone B | C21 H26 O9 | 422.15 | 12.172 | 15,882,130,810 |
| | (1R,3R,4S,6S,8R,9R,10S,11S)- | | | | |
| | 9-(acetyloxy)-8-hydroxy- | | | | |
| | 3,8-dimethyl-12- | | | | |
| | methylidene-7,13-dioxo-5,14 | | | | |
| | dioxatricyclo [9.3.0.04,6]tetradecan- | | | | |
| | 10-yl 2-methylprop-2-enoate | | | | |
| A6 | Yangonin | C15 H14 O4 | 258.08 | 12.172 | 1,244,465,484 |
| A7 | Flavone, 2-Phenyl-4H-1- | C15 H10 O2 | 222.06 | 12.172 | 93,629,315 |
| | benzopyran-4one, 2- | | | | |
| | Phenylchromone | | | | |
| A8 | α-Lapachone, 3,4-Dihydro- | C15 H14 O3 | 242.09 | 12.28 | 27,091,250 |
| | 2,2-dimethyl-2H- | | | | |
| | naphtho[2,3-b]pyran-5,10- | | | | |
| | dione | | | | |
| A9 | Murrangatin 8-(1,2- | C15 H16 O5 | 276.09 | 12.391 | 152,423,283 |
| | dihydroxy-3-methylbut-3-en- | | | | |
| | 1-yl)-7-methoxy-2H- | | | | |
| | chromen-2-one | | | | |
| A10 | (1R,4aS,5R,8aS)-5-(5- | C20 H34 O3 | 322.25 | 14.517 | 4,378,871 |
| | hydroxy-3-methylpentyl)- | | | | |
| | 1,4a-dimethyl-6- | | | | |
| | methylidene- | | | | |
| | decahydronaphthalene-1- | | | | |
| | carboxylic acid | | | | |

TABLE 4

| Compound | | | Calc. | RT | Area | Area |
|---|---|---|---|---|---|---|
| No. | Compound name | Formula | MW | [min] | Ext. A | Ext. C |
| AC1 | DL-Carnitine | C7 H15NO3 | 161.10 | 0.708 | 386,595,363 | 188,858.350 |
| AC2 | Valine | C5 H11NO2 | 117.07 | 0.823 | 299,719,731 | 762,343,180 |
| AC3 | L-Norleucine | C6 H13NO2 | 131.09 | 1.986 | 105,168,196 | 776,052,439 |
| AC4 | (2R,3S,4S,5R,6R)- | C14 H20 O6 | 284.12 | 6.62 | 4,766,900,780 | 3,965,248,469 |
| | 2-(hydroxymethyl)-6- | | | | | |
| | (2-phenylethoxy)oxane- | | | | | |
| | 3,4,5-triol | | | | | |
| AC5 | Genistin | C21H20O1O | 432.10 | 7.887 | 46,850,468 | 19,272,245 |
| AC6 | Kuromanin | C21H20O11 | 448.10 | 8.206 | 7,728,621 | 14,178,412 |
| AC7 | NP-020760 | C21 H38 O8 | 418.25 | 8.509 | 169,216,326 | 286,281,319 |
| AC8 | NP-008952 | C12 H20 O4 | 228.13 | 10.473 | 5,133,756 | 14,116,256 |
| AC9 | Pelargonidin, | C15 H10 O5 | 270.05 | 10.588 | 1,302,387,012 | 1,672,611,309 |
| | (3,4′,5,7- | | | | | |
| | Tetrahydroxyflavylium) | | | | | |
| AC10 | Aflatoxin B1 | C17 H12 O6 | 312.06 | 11.234 | 1,468,738 | 6,638,291 |
| AC11 | 2,20-dihydroxy-13- | C25 H32 O8 | 460.20 | 12.586 | 3,232,602 | 14,795,380 |
| | methoxy- | | | | | |
| | 4,7,17,22,22- | | | | | |
| | pentamethy | | | | | |
| | 15,10,21,23tetraoxahexacyclo | | | | | |
| | [18.2.1.0Aa,Aaa•.0a, | | | | | |
| | Aaa.0a,Aaa.0a,AaÂ | | | | | |
| | tricosa-6(14),7,12-trien-11-one | | | | | |
| AC12 | (2R,3R,3aR,5R)- | C22 H26 O7 | 402.16 | 12.843 | 125,072,984 | 114,632,747 |
| | 5,7-dimethoxy-2- | | | | | |
| | (7-methoxy-2H-1,3- | | | | | |
| | benzodioxol-5-yl)- | | | | | |
| | 3-methyl-3a-(prop- | | | | | |
| | 2-en-1-yl)2,3,3a,4,5,6- | | | | | |
| | hexahydro-1- | | | | | |
| | benzofuran-6-one | | | | | |
| AC13 | Octadecanamine | C18 H39 N | 269.30 | 13.341 | 6,573,660 | 3,819,614 |
| AC14 | Oleamide | C18 H35NO | 281.27 | 15.712 | 2,177,872,750 | 1,919,921,186 |

TABLE 5

| Compound | | | Calc. | RT | Area |
|---|---|---|---|---|---|
| No. | Compound name | Formula | MW | [ min] | Ext. C |
| C1 | 1-O-(3,4,5- | C16H22O10 | 374.12 | 3.875 | 148,046,091 |
| | Trimethoxybenzoyl)- | | | | |
| | beta-L-galactopyranose | | | | |
| C2 | 3-Methoxybenzaldehyde | C8 H8 O2 | 136.05 | 4.342 | 46,695,749 |
| C3 | (1S,4aS,7aR)-7-methyl-1- | C21H30O13 | 490.16 | 5.707 | 29,114,110 |
| | {[(2S,3R,4S,5S,6R)- | | | | |
| | 3,4,5-trihydroxy-6- | | | | |
| | (hydroxymethyl)oxan- | | | | |
| | 2-yl]oxy}-4a- | | | | |
| | {[(2S,3R,4R,5R,6S)- | | | | |
| | 3,4,5-trihydroxy-6- | | | | |
| | methyloxan-2-yl]oxy}- | | | | |
| | 1H,4aH,5H,7aH- | | | | |
| | cyclopenta [c]pyran-5- | | | | |
| | one | | | | |
| C4 | 3-(4-hydroxyphenyl)-7- | C22H22O10 | 446.12 | 6.186 | 8,225,089 |
| | methoxy-5- | | | | |
| | {[(3R,4S,5S,6R)-3,4,5- | | | | |
| | trihydroxy-6- | | | | |
| | (hydroxymethyl)oxan- | | | | |
| | 2-yl]oxy}-4H- | | | | |
| | chromen-4-one | | | | |
| C5 | 1-(3-methoxy-4- | C21H30O12 | 474.17 | 6.236 | 73,826,648 |
| | {[(2S,3R,4S,5S,6R)- | | | | |
| | 3,4,5-trihydroxy-6- | | | | |
| | ({[(2R,3R,4R,5R,6S)- | | | | |
| | 3,4,5-trihydroxy-6- | | | | |
| | methy-loxan-2- | | | | |
| | yl]oxy}methyl)oxan-2- | | | | |
| | yl]oxy} phenyl)ethan- | | | | |
| | 1-one | | | | |
| C6 | (2R,3R,4S,5S,6R)-2- | C16 H28 O7 | 332.18 | 6.99 | 399,856,237 |
| | {[(2E,6R)-6-hydroxy- | | | | |
| | 2,6-dimethylocta-2,7- | | | | |
| | dien-1-yl]oxy}-6- | | | | |
| | (hydroxymethyl)oxane- | | | | |
| | 3,4,5-triol | | | | |
| C7 | Solanid-5-en-3-yl 6- | C45H73NO15 | 867.49 | 9.292 | 5,989,523 |
| | deoxy-alpha-L- | | | | |
| | mannopyranosyl-(1-2)- | | | | |
| | [beta-D- | | | | |
| | glucopyranosyl-(1-3)]- | | | | |
| | beta-D- | | | | |
| | mannopyranoside | | | | |
| C8 | NP-008274 | C15 H20 O4 | 264.13 | 10.291 | 6,514,510 |
| C9 | Formononetin | C16 H12 O4 | 268.07 | 11.904 | 11,361,966 |
| C10 | Spiroxamine | C18H35NO2 | 297.26 | 12.983 | 24,206,021 |

TABLE 6

| Compound | | | Calc. | RT | Area | Area |
|---|---|---|---|---|---|---|
| No. | Compound name | Formula | MW | [min] | Ext. A | Ext. B |
| AB1 | Phloroglucinol | C6 H6 O3 | 126.03 | 0.707 | 41,675,260 | 276,902,261 |
| AB2 | Caffeic acid | C9 H8 O4 | 180.04 | 5.467 | 37,440,395 | 117,292,952 |
| AB3 | (5S,6S)-5-hydroxy- | C14H16O4 | 248.10 | 6.563 | 37,379,312 | 81,788,995 |
| | 4-methoxy-6-(2-phe- | | | | | |
| | nylethyl)-5,6-dihyd- | | | | | |
| | ro-2H-pyran-2-one | | | | | |
| AB4 | Quercetin-3 ß-D- | C21H20O12 | 464.09 | 7.009 | 1,944,716,030 | 260,845,893 |
| | glucoside | | | | | |
| AB5 | 2-(3,4-dihydrox- | C20H18O11 | 434.08 | 7.464 | 66,392,856 | 23,856,352 |
| | yphenyl)-5,7-dihy- | | | | | |
| | droxy-3-{[(2S,3R,4S,5S)- | | | | | |
| | 3,4,5-trihy- | | | | | |
| | droxyoxan-2-yl]oxy}- | | | | | |
| | 4H-chromen-4-one | | | | | |
| AB6 | 5,7-dihydroxy-2-(4- | C22H22O12 | 478.11 | 8.694 | 1,997,194 | 26,272,176 |
| | hydroxy-3-{[(2S,3R,4S,5S,6R)- | | | | | |
| | 3,4,5- | | | | | |
| | trihydroxy-6-(hydro- | | | | | |
| | xymethyl)oxan-2- | | | | | |
| | yl]oxy}phenyl)-3- | | | | | |
| | methoxy-4H- | | | | | |
| | chromen-4-one | | | | | |
| AB7 | 4-Hydroxycoumarin | C9 H6 O3 | 162.03 | 9.875 | 2,384,008,330 | 5,413,658,494 |
| AB8 | 2-Hydroxycinnamic | C9 H8 O3 | 164.04 | 11.409 | 78,128,321 | 271,611,189 |
| | acid | | | | | |
| AB9 | 19-norandrostenedion | C18 H24 O2 | 272.17 | 12.582 | 6,732,704 | 24,425,895 |

TABLE 7

| Compound | | | Calc. | RT | Area |
|---|---|---|---|---|---|
| No. | Compound name | Formula | MW | [min] | Ext. B |
| B1 | 6-Pentyl-2H-pyran-2-one | C10 H14 O2 | 166.09 | 5.466 | 367,183,571 |
| B2 | Myricetin 3-O-beta-D-galactopyranoside | C21 H20 O13 | 480.08 | 6.353 | 54,473,499 |
| B3 | 5,7-Dihydroxy-2-(4-hydroxyphenyl)- | C27 H30 O15 | 594.15 | 7.388 | 224,327,842 |
| | 4-oxo-4H-chromen-3-yl6-O-(6- | | | | |
| | deoxyhexopyranosyl)hexopyranoside | | | | |
| B4 | NP-003191 | | | | |
| B5 | (2R,3S,4S,5R,6R)-2-({[(2R,3R,4R, | C21 H36 O10 | 448.23 | 8.926 | 200,715,620 |
| | 5S)-3,4-dihydroxy-5-(hydroxy | | | | |
| | methyl) oxolan-2-yl]oxy}methyl)-6- | | | | |
| | {[(2E)-3,7-dimethylocta-2,6-dien-1- | | | | |
| | yl]oxy}oxane-3,4,5-triol | | | | |
| B6 | 2,6-Di-tert-butyl-1,4-benzoquinone | C14 H20 O2 | 220.14 | 9.197 | 116,434,233 |
| B7 | 4-Methoxycinnamaldehyde | C10 H10 O2 | 162.06 | 10.772 | 47,596,601 |
| B8 | Sakuranetin | C16 H14 O5 | 286.08 | 12.73 | 23,909,168 |
| B9 | Estriol | C18 H24 O3 | 288.17 | 13.532 | 22,924,076 |
| B10 | Testosterone | C19 H28 O2 | 288.20 | 13.585 | 40,718,111 |
| B11 | Nootkatone, ((4,4a,5,6,7,8- | C15 H22 O | 218.16 | 14.248 | 706,868,081 |
| | hexahydronaphthalen-2(3H)-one) | | | | |
| B12 | 2,4-Dimethylbenzaldehyde | C9 H10 O | 134.07 | 14.304 | 80,005,955 |

TABLE 8

| Compound | | | Calc. |
|---|---|---|---|
| No. | Compound name | Formula | MW |
| ABC1 | Betaine | C5 H11NO2 | 117.07 |
| ABC2 | Proline | C5 H9 N O2 | 115.06 |
| ABC3 | L-Pyroglutamic acid | C5 H7 N O3 | 129.04 |
| ABC4 | L-Phenylalanine | C9 H11NO2 | 165.07 |
| ABC5 | (1r,3R,4s,5S)-4-{[(2E)-3- | C16 H18O9 | 354.09 |
| | (3,4-dihydroxyphenyl) | | |
| | prop-2-enoyl]oxy}-1,3,5- | | |
| | trihydroxy cyclohexane-1- | | |
| | carboxylic acid; | | |
| ABC6 | (1S,3R,4R,5R)-1,3,4- | C17 H2O O9 | 368.11 |
| | trihydroxy-5-{[(2E)-3-(4- | | |
| | hydroxy-3- | | |
| | methoxyphenyl)prop-2- | | |
| | enoyl]oxy}cyclohexane-1- | | |
| | carboxylic acid; | | |
| ABC7 | Scutellarin | C21H18O12 | 462.07 |
| ABC8 | 4,5-dicaffeoylquinic acid | C25H24O12 | 516.12 |
| | (Isochlorogenic acid C) OK | | |
| | 1R,3R,4S,5R)-3,4- | | |
| | bis({[(2E)-3-(3,4- | | |
| | dihydroxyphenyl)prop-2- | | |
| | enoyl[oxy})-1,5-dihydroxy | | |
| | cyclohexane-1-carboxylic | | |
| | acid | | |
| ABC9 | 5-hydroxy-2-(4- | C22H22O11 | 462.11 |
| | hydroxyphenyl)-6- | | |
| | methoxy- | | |
| | 7{[(2S,3R,4S,5S,6R)-3,4,5- | | |
| | trihydroxy-6- | | |
| | (hydroxymethyl) oxan-2- | | |
| | yl]oxy}-4H-chromen-4-one | | |
| ABC10 | Eriodictyol[(S)-2-(3,4- | C15 H12 O6 | 288.06 |
| | dihydroxyphenyl)-5,7- | | |
| | dihydroxy-2,3- | | |
| | dihydrochromen-4-one] or | | |
| | 3′,4′,5,7-tetrahydroxy | | |
| | flavanone or 2,3- | | |
| | dihydroluteolin, | | |
| ABC11 | Cyanidin | C15 H10 O6 | 286.04 |
| ABC12 | Diosmetin | C16 H12 O6 | 300.06 |
| ABC13 | (±)13-HpODE | C18 H32 O4 | 312.23 |
| ABC14 | 5,7-dihydroxy-2-(3- | C18 H16 O8 | 360.08 |
| | hydroxy-4 | | |
| | methoxyphenyl)-3,6- | | |
| | dimethoxy-4H-chromen-4- | | |
| | one | | |
| ABC15 | Glycitein | C16 H12 O5 | 284.06 |
| ABC16 | Nobiletin | C21 H22 O8 | 402.13 |
| ABC17 | Bis(methylbenzylidene)sorbitol | C22 H26 O6 | 386.17 |
| ABC18 | Aflatoxin B2 | C17 H14 O6 | 314.08 |
| ABC19 | 13,14-dihydro-15-keto- | C16 H26 O5 | 298.17 |
| | tetranor Prostaglandin E2 | | |
| ABC20 | 12-Oxophytodienoic acid | C18 H28 O3 | 292.20 |
| ABC21 | (2S,4aS,6aS,6bR,10S,12aS,14bS)- | C30 H46 O4 | 470.34 |
| | 10-hydroxy- | | |
| | 2,4a,6a,6b,9,9,12a- | | |
| | heptamethyl-13-oxo- | | |
| | 1,2,3,4,4a,5,6,6a,6b,7,8,8a,9,10,11,12,12a,12b,13,14b- | | |
| | icosahydropicene-2- | | |
| | carboxylic acid | | |
| ABC22 | 9-Oxo-10(E),12(E)- | C18 H30O3 | 294.2191 |
| | octadecadienoic acid | | |
| ABC23 | α-Linolenoyl ethanolamide | C20H35NO2 | 321.2664 |
| ABC24 | Linoleoyl ethanolamide | C20 H3 NO2 | 323.2819 |
| ABC25 | Arachidonic acid | C20 H32 O2 | 304.2398 |
| ABC26 | 5-(1,2,4a,5-tetramethyl-7- | C20 H32 O3 | 320.2348 |
| | oxo-1,2,3,4,4a,7,8,8a- | | |
| | octahydronaphthalen-1- | | |
| | yl)-3-methylpentanoic acid | | |
| ABC27 | 8Z,11Z,14Z-Eicosatrienoic | C20 H34 O2 | 306.2555 |
| | acid | | |
| ABC28 | 2,4-Xylidine | C8 H11 N | 121.0893 |
| ABC29 | Quercetin | C15 H10 O7 | 302.04 |
| ABC30 | Sphingosine | C18 H37N O2 | 299.28 |
| ABC31 | Rhamnetin (2-(3,4- | C16 H12 O7 | 316.05 |
| | dihydroxyphenyl)-3,5- | | |
| | dihydroxy-7-methoxy-4h- | | |
| | chromen-4-one | | |
| ABC32 | (1S,4S,5R,10S,13S,17S,19S,20R)- | C30 H46 O3 | 454.34 |
| | 10-hydroxy- | | |
| | 4,5,9,9,13,19,20- | | |
| | heptamethyl-24- | | |
| | oxahexacyclo | | |
| | [15.5.2.0¹,¹⁸.0⁴,¹⁷.0⁵,¹⁴.0⁸,¹³] | | |
| | tetracos-15-en-23-one | | |

| Compound | RT | Area | Area | Area |
|---|---|---|---|---|
| No. | [min] | Ext. A | Ext. B | Ext. C |
| ABC1 | 0.694 | 3,530,740,114 | 774,970,563 | 1,351,792,437 |
| ABC2 | 0.759 | 2,596,224,600 | 4,829,925,973 | 476,675,060 |
| ABC3 | 0.819 | 120,309,068 | 535,269,526 | 711,595,064 |
| ABC4 | 3.256 | 46,652,027 | 119,879,831 | 229,319,024 |
| ABC5 | 5.054 | 717,332,175 | 1,566,476,228 | 22,570,076 |
| ABC6 | 6.232 | 6,050,155 | 125,783,387 | 4,361,948 |
| ABC7 | 7.162 | 9,544,332 | 3,987,959 | 399,151,403 |
| ABC8 | 7.949 | 717,332,175 | 1,566,476,228 | 22,570,076 |
| ABC9 | 8.116 | 5,934,396 | 6,322,103 | 48,852,430 |
| ABC10 | 9.278 | 3,445,353 | 54,410,748 | 3,115,742 |
| ABC11 | 9.509 | 365,449,990 | 1,847,774,673 | 6,004,521,060 |
| ABC12 | 10.908 | 1,210,096,313 | 778,882,279 | 3,374,118,743 |
| ABC13 | 11.548 | 29,794,300 | 151,854,000 | 29,790,793 |
| ABC14 | 11.704 | 1,918,047 | 15,810,934 | 5,900,261 |
| ABC15 | 12.836 | 1,871,318,710 | 2,048,571,322 | 16,749,470 |
| ABC16 | 12.864 | 28,776,385 | 8,388,672 | 28,689,196 |
| ABC17 | 12.906 | 27,466,171,962 | 4,220,658,636 | 27,902,714,014 |
| ABC18 | 13.001 | 2,736,807,427 | 874,589,672 | 9,950,100 |
| ABC19 | 13.965 | 50,183,155 | 6,591,428 | 16,587,611 |
| ABC20 | 14.178 | 188,626,030 | 421,917,113 | 379,074,020 |
| ABC21 | 14.492 | 10,734,543 | 41,053,407 | 164,719,754 |
| ABC22 | 14.536 | 836,727,031 | 1,137,604,890 | 206,890,918 |
| ABC23 | 14.584 | 96,331,653 | 144,983,358 | 22,559,2473 |
| ABC24 | 15.011 | 382,279,845 | 622,398,515 | 292,070,982 |
| ABC25 | 15.208 | 421,592,785 | 746,792,985 | 756,456,211 |
| ABC26 | 15.372 | 932,482,943 | 272,485,224 | 2,315,762,663 |
| ABC27 | 15.482 | 71,268,301 | 45,589,288 | 65,609,194 |
| ABC28 | 16.933 | 536,746,096 | 140,381,883 | 414,932,253 |
| ABC29 | 8.303 | 906,000,000 | 370,000,000 | 9,436,007 |
| ABC30 | 13.293 | 115,704,693 | 33,337,083 | 3,739,707 |
| ABC31 | 12.088 | 3,999,606 | 487,756,323 | 2,123,057 |
| ABC32 | 14.413 | 6,081,044 | 38,975,281 | 345,271,325 |

TABLE 9

| Compound | | | Calc. | RT | Area | Area |
|---|---|---|---|---|---|---|
| No. | Compound name | Formula | MW | [min] | Ext. B | Ext. C |
| BC1 | Trigonelline (N- | C7 H7 NO2 | 137.04 | 0.742 | 262,043,732 | 106,297,400 |
| | Methylnicotinate) | | | | | |
| BC2 | 4-(4-hydroxy-2,6,6- | C19 H32 O8 | 388.20 | 5.857 | 1,9167,772 | 488,977,805 |
| | trimethyl-3- | | | | | |
| | {[(2R,3R,4S,5S,6R)-3,4,5- | | | | | |
| | trihydroxy-6- | | | | | |
| | (hydroxymethyl)oxan-2- | | | | | |
| | yl]oxy}cyclohex-1-en-1- | | | | | |
| | yl)butan-2-one | | | | | |
| BC3 | 5-(6-hydroxy-6- | C13 H22 O3 | 226.15 | 5.868 | 23,817,617 | 27,604,558 |
| | methyloctyl)-2,5- | | | | | |
| | dihydrofuran-2-one | | | | | |
| BC4 | Rutin | C27H30O16 | 610.15 | 6.884 | 715,750,931 | 7,483,570 |
| BC5 | [(2R,3S,4S,5R,6R)-6-[2- | C23 H26 O11 | 478.14 | 7.165 | 9163,930 | 382,007,136 |
| | (3,4-dihydroxyphenyl) | | | | | |
| | ethoxy]-3,4,5- | | | | | |
| | trihydroxyoxan-2-yl]methyl | | | | | |
| | (2E)-3-(3,4- | | | | | |
| | dihydroxyphenyl)prop-2- | | | | | |
| | enoate | | | | | |
| BC6 | Trifolin | C21H20O11 | 448.09 | 7.714 | 249,579,025 | 14,131,492 |
| BC7 | (2S,3S,4S,5R,6S)-6-{[5,7- | C21H18O12 | 462.07 | 8.204 | 1,664,311 | 81,408,493 |
| | dihydroxy-2- | | | | | |
| | (4-hydroxyphenyl)-4-oxo- | | | | | |
| | 4H-chromen-3-yl]oxy}-3,4,5- | | | | | |
| | trihydroxyoxane-2- | | | | | |
| | carboxylic acid | | | | | |
| BC8 | Chrysin | C15 H10 O4 | 254.05 | 9.082 | 2,016,251 | 28,745,233 |
| BC9 | Isophorone | C9 H14 O | 138.10 | 10.381 | 1,644,808,897 | 4,765,689 |
| BC10 | (2R,5R,6R)-3-[(1E,3E)- | C14 H22 O4 | 254.15 | 10.67 | 201,206,965 | 8,524,318 |
| | hepta-1,3-dien-1-y]-5,6- | | | | | |
| | dihydroxy-2- | | | | | |
| | (hydroxymethyl)cyclohexan- | | | | | |
| | 1-one | | | | | |
| BC11 | Carvone | C10 H14 O | 150.10 | 11.434 | 2,305,669,446 | 8,898,044 |
| BC12 | 3-Methoxy prostaglandin | C21 H38 O6 | 386.26 | 12.769 | 214,114 | 20,526,597 |
| | F1α | | | | | |
| BC13 | 1,4-dihydroxy-1,4-dimethyl- | C15 H24 O3 | 252.17 | 12.853 | 324,663,217 | 5,935,783 |
| | 7-(propan-2-ylidene)- | | | | | |
| | decahydroazulen-6-one | | | | | |
| BC14 | 5-hydroxy-6,7-dimethoxy-2- | C17 H14 O5 | 298.08 | 14.032 | 272,839,318 | 2,225,655 |
| | phenyl-4H-chromen-4-one | | | | | |
| BC15 | Bicyclo Prostaglandin E2 | C20 H30O4 | 334.21 | 14.454 | 17,867,376 | 27,812,813 |
| BC15 | 2,4,5-Trimethylaniline | C9 H13N | 135.10 | 16.8 | 83,080,637 | 56,870,246 |

As demonstrated in the appended examples, the hydroethanolic extract was successfully used in several patients to treat and/or ameliorate the conditions and/or symptoms associated with a disease they were suffering from. It has been very surprisingly found that the hydroethanolic extract of Vernonanthura nudilora, Baccharis articulata, and Plantago major were very useful for improvement in symptoms of patients suffering from a variety of diseases or disorders of the central nervous system, such as autism, schizophrenia, epilepsy, and even the retarded development observed in trisomy 21 genetic disease patients.

Therefore, as detailed above, provided herein a method for treating of a neurological and/or psychiatric disorder. As generally used herein, the neurological and/or psychiatric disorder is preferably a disease or disorder classified in a subclass 06 or 08 of the 11^{th} revision of World Health Organization International Classification of Diseases (herein: “ICD-11”), although diseases from other classes wherein the involvement of nerves or nervous system is observed may be responsive to the treating with the extract as described herein, e.g., the diseases classified in a subclass 07, 09, 10, or in a subclass 17. In some particular embodiments, the neurological and/or psychiatric disorder may be selected from the group consisting of neurodevelopmental disorders, schizophrenia or other primary psychotic disorders, catatonia, mood disorders, anxiety or fear-related disorders, obsessive-compulsive or related disorders, disorders specifically associated with stress, dissociative disorders, feeding or eating disorders, elimination disorders, disorders of bodily distress or bodily experience, disorders due to substance use or addictive behaviors, impulse control disorders, disruptive behavior or dissocial disorders, personality disorders and related traits, paraphilic disorders, factitious disorders, neurocognitive disorders, and symptoms thereof primary or secondary to an accompanied disease or condition. Additionally, in some further particular embodiments, the neurological and/or psychiatric disorder may be selected from the group consisting of movement disorders, disorders with neurocognitive impairment, multiple sclerosis or other white matter disorders, epilepsy or seizures, headache disorders, cerebrovascular diseases, spinal cord disorders excluding trauma, motor neuron diseases or related disorders, disorders of nerve root, plexus or peripheral nerves, diseases of neuromuscular Junction or muscle, and cerebral palsy. Further additionally, the neurological and/or psychiatric disorder may be selected from sleep-wake disorders, diseases of visual system excluding structural, diseases ear or mastoid process excluding structural, and conditions related to sexual health, all as defined in subclasses 07, 09, 10, and 17, respectively, of the ICD-11.

In some currently preferred embodiments, the neurological and/or psychiatric disorder is a neurodevelopmental disorder, an anxiety or fear-related disorder, a primary psychotic disorder, or a seizures disorder. In currently preferred embodiments, the developmental disorder may be an autism spectrum disorder, e.g., Asperger's syndrome, an attention deficit hyperactivity disorder, or Down Syndrome-associated development retardation. The anxiety or fear-related disorder may be a primary psychotic disorder, e.g., schizophrenia. The seizures disorder may be an epilepsy, or a hemiplegia.

The diseases and conditions and their respective symptoms, pathophysiology, and prognosis are generally known to the practicing physicians in their respective field of expertise.

For example, autism spectrum disorder is currently defined as a group of neurodevelopmental abnormalities with broadly varying degrees and manifestations that usually begin in early childhood and are characterized by problems in social communication and interaction, along with behavioral problems such as restricted interests and repetitive behaviors. Asperger's Syndrome, presently considered to be a form of ASD, is a developmental disorder. Young people with Asperger's Syndrome have a difficult time relating to others socially and their behavior and thinking patterns may be rigid and repetitive, and they have trouble understanding social situations and subtle forms of communication.

Trisomy 21, commonly known as Down Syndrome (or Down's syndrome), is a genetic disorder caused by the presence of all or part of a third copy of chromosome 21, resulting, inter alia, in accumulation of amyloid proteins overexpressed due to presence of an additional chromosome. It is usually associated with mental impairment, developmental delays, and characteristic physical features.

Anxiety disorders are the most common of mental disorders and may be present as such or is conjunction to other disorders, or mental states. Anxiety is a normal reaction to stress. Mild anxiety may increase the alertness in stressful situations. Abnormal anxiety is a complex cognitive, affective, physiological, and behavioral response system which involves panic, excessive fear, which is out of proportion to the situation and age, and may significantly hinder the ability of the affected person to function normally. Anxiety disorders affect nearly 30% of adults at some point in their lives. Many types of anxiety disorders have been recognized, including generalized anxiety disorder, panic, phobias, and many others. Related conditions include post-traumatic stress disorder, obsessive-compulsive disorder, and several other psychiatric conditions. Physiologically, anxiety involves amygdala and limbic system structures in the prefrontal cortex regions of the brain, and prefrontal-limbic activation abnormalities may be reversed with psychological or pharmacologic interventions. Noradrenaline, serotonin, dopamine, and gamma-aminobutyric acid have been implicated in the emergence and modulation of anxiety pathophysiology. The autonomic nervous system, especially the sympathetic nervous system, mediates most of the symptoms. Conventionally, the anxiety disorders are treated with tranquilizers, such as benzodiazepines, as well as neurotransmitter reuptake inhibitors, tricyclic antidepressants, and also beta-blockers to control the manifestations of anxiety.

Schizophrenia is a serious mental disorder in which people interpret reality abnormally. Schizophrenia involves a range of cognitive, behavioral and emotional problems. The underlying mechanisms of schizophrenia are yet poorly understood, but it involves dopaminergic neurons in the central nervous system. Signs and symptoms vary, but usually involve delusions, hallucinations, disorganized speech, and reflect an impaired ability to function. Treatment has traditionally been neuroleptics and tranquilizers. Schizophrenia may also be accompanied by other psychiatric disorders or symptoms thereof.

Alternating hemiplegia of childhood is a neurological disorder that requires lifelong care. Around 75% of people with alternating hemiplegia of childhood have a mutation in a specific gene, ATP1A3. This gene mutation is usually spontaneous, meaning it rarely runs in families. Symptoms begin before 18 months of age and include recurring episodes of paralysis that may attack one side of the body or both sides at once. Episodes may also include painful muscle stiffness and uncontrollable eye movements. Symptoms can last for minutes to days and are relieved with sleep. Up to half of children with AHC eventually develop epilepsy.

As used herein, the term “treating”, “treatment”, and the like, are used interchangeably to refer to improvement of symptoms or intervening in a process that is an underlying cause for the disease or disorder being “treated”. The treatment includes ameliorating the symptoms, inhibition or delaying disease progression, or reversal thereof. The treatment also includes delaying an onset of symptoms that a likely to appear in a patient suffering from the same disease or condition without any treatment. The terms also encompass the prophylactic treatment, e.g., prevention of an onset of a symptom or a condition, and may thereby also refer to reducing the prevalence of said symptom or condition or reducing the likelihood that the symptom or condition will emerge during the “treatment”. The terms “patient”, “subject”, and the like, as used herein refers generally to a human subject afflicted by the disease or disorder.

The efficiency of treating of a disease or a disorder may be assessed as known in the art, e.g., with clinical trials. Preferably, the clinical trials are controlled clinical trials. Depending on the purpose of the clinical study and the objectives thereof, the control group to measure an effect of a clinical trial may be a group receiving an inactive composition, an unrelated plant extract, a supportive treatment, or a know standard of care. The clinical study is usually a randomized clinical study, wherein the control group receives one of the control treatments, and the treatment groups receive varying doses of the compositions comprising hydroethanolic extracts as described generally herein. The tested doses' range may be determined in a previous pilot clinical study, based on a pre-clinical data, and otherwise procured as known in the art. The clinical study may have one or more of primary end points, i.e., measurable outcomes, and may further have one or more of secondary end points, i.e., further measurable outcomes. The clinical study may be considered successful if at least one primary and/or at least one secondary outcomes are reached, that is, the measurable outcomes of a treatment group is statistically significantly different and within the predefined range of difference in comparison to the measurable outcome of a control group. The clinical studies may enroll sufficient number of patients, the number being controlled by the outcomes sought and the anticipated differences, to meet the required statistical power. The enrolled patients must meet the inclusion criteria on the disease or disorder, the condition, and/or the symptoms, and must not have any of the exclusion criteria defined to disallow certain patients that might not benefit from the treatment or may be in danger developing a side effect or having otherwise compromised medical safety as the result of the treatment.

Treating of a patient in need thereof as disclosed herein entails administering to the patient an effective amount of the hydroethanolic extract. As seen in the appended examples, the amount required to treat chronic or conditions, such as neurodevelopment disorder, is rather small, e.g., between 4 and 20 mL of 1:8 diluted extract (e.g., with water), but the extract could also be used in rather high amounts in the treatment of a primary psychotic disorders, e.g., up to 60 mL of diluted extract. The amounts of administered dose may be recalculated on the dry basis, i.e., the amount of dried plant-derived content after evaporating the solvent of the hydroethanolic extract. The dose is preferably expressed based on the dry basis of the extract. The doses (i.e., therapeutically effective amounts) may vary between 0.008 mL/kg (kg-weight of the patient) and 2 mL/kg of the diluted 1:8 hydroethanolic extracts, i.e., between 0.001 mL/kg and 0.25 mL/kg of concentrated mL, which means between 0.033 mg/kg (of dry mass/kg of patient) and 8.6 mg/kg (of dry mass/kg of patient). Preferably, dosages (i.e., therapeutically effective amounts) may vary between 0.048 mL/kg (kg-weight of the patient) and 1 mL/kg of the diluted 1:8 hydroethanolic extracts, i.e., between 0.006 mL/kg and 0.125 mL/kg of concentrated mL, which means between 0.2 mg/kg (of dry mass/kg of patient) and 4.3 mg/kg (of dry mass/kg of patient). Thus, in some embodiments, the dose may vary between 5 and 400 mg per day. The dose may also be between about 10 and about 120 mg per day for a patient suffering from a neurodevelopment disorder, and may be between 100 and 300 mg per day for a patient suffering from a primary psychotic disorder.

Specific doses for specific diseases or disorders may be adjusted depending on the severity of the condition, the patient factors, e.g., weight, age, comorbidities, etc., and the desired treatment outcome.

The daily doses as described herein may be administered in a variety of regimens, depending on the patient mental status, general health condition, and desired outcome. The regimens include once daily administration, that is, the whole daily dose is administered at once every day; twice daily administration, that is, the total daily dose is divided into two preferably equal doses and administered in a reasonable interval, preferably every 12 hours; three time a day, that is, the total daily dose is divided into three preferably equal doses and administered in a reasonable interval, preferably every 8 hours; or four times a day, that is, the total daily dose is divided into four preferably equal doses and administered in a reasonable interval, preferably every 6 hours.

Preferably, the hydroethanolic extracts as described herein and the compositions comprising same may be administered perorally, with or without food, preferably, without food. However, the extracts may under certain conditions be administered also by intravenous route, intramuscular route, or intraperitoneal route.

In some further embodiments, the method as described herein further comprises administering to said subject an additional therapeutic agent. The additional therapeutic agents may be selected according to the needs of a specific patient and under proper medical judgement of the treating doctor. The additional therapeutic agents may address symptoms not adequately controlled by the hydroethanolic extract as described herein, or may be used as a primary treatment, with the hydroethanolic extract as described herein serving as an additional (e.g., adjuvant) treatment.

In some further embodiments, the hydroethanolic extract may be provided in a composition comprising same. For administration by a parenteral route, the extract may be provided as a liquid for injection. Preferably, e.g., for intravenous administration, the liquid comprising the extract is diluted in a large volume, e.g., of a physiological solution as used in medicine. For this purpose, the hydroethanolic extract (or a dried form thereof) may be combined with inert ingredients, e.g., pharmaceutical excipients. The skilled artisan is referred to the Remington's Pharmaceutical Sciences, 18^{th} edition, 1990, ISBN 9780912734040, or to a comparable textbook, for the principles of formulation of dosage forms.

The hydroethanolic extract may be formulated in a liquid dosage form. The liquid dosage form may be the original hydroethanolic extract, in which case it will be in a form or a tincture. The concentration of the dried basis of the tincture may be adjusted by dilution, to enable more accurate dosing. The extract may also be in a form of a diluted extract, with the dilution of more than 1:5. The diluted extract may contain the same solvent as the original hydroethanolic extract, or may have a higher amount of water. The diluted extract may further comprise preservatives, if the concentration of ethanol in the diluted extract is below 20% by volume.

The hydroethanolic extract may be further formulated into a medicated syrup, e.g., by mixing the original tincture with a predetermined amount of a syrup. To maintain the hydroethanolic composition, the tincture may be further formulated into an elixir, e.g., by mixing the original tincture with a predetermined amount of a syrup diluted with ethanol.

The hydroethanolic extract, particularly dried or lyophilized hydroethanolic extract, may be formulated into a capsule, e.g., a gelatin capsule, or hypromellose capsule. the liquid hydroethanolic extract may be adsorbed onto a carrier, and then carefully evaporated till dryness, to furnish a powder suitable for filling a capsule. The dried extract or adsorbed extract may further be compressed into a tablet, as known in the art. The tablets or capsules may be further coated, e.g., with a protective coating, and packaged into a suitable container, such as a bottle or a blister-pack.

Inactive ingredients, suitable for the dosage forms as described herein, may be found in the Handbook of Pharmaceutical Excipients, 9^{th} edition, 2020, ISBN 9780857113757, or an equivalent text as known to the skilled artisan.

In a further aspect provided herein a composition comprising a hydroethanolic plant extract or any fraction thereof, for use in a method as generally disclosed herein. In a yet further aspect provided herein a dosage form comprising the composition as disclosed herein, preferably the dosage form being selected from the group consisting of a tincture, a syrup, an elixir, a capsule, and a tablet.

Various other features according to the invention as described herein for the aspect of the methods of treating of conditions are applicable mutatis mutandis to compositions and/or dosage forms for use in these methods according to the teachings herein. The terms “composition” and the like as used herein interchangeably to hydroethanolic plant extracts as described herein, and to compositions comprising at least a fraction of the hydroethanolic extracts, e.g., compositions ready for administering to a patient in need thereof, or to the dosage forms as described herein.

The herein described preferred embodiments provided herein demonstrating some of the embodiments of the present disclosure are provided to better understand the present disclosure, which however does not limit the invention in any respect. Variants and equivalents may be readily envisaged by the skilled artisan; the invention therefore encompasses all these variations and equivalents.

It must also be noted that, as used in this specification and the appended claims: all scientific and technical terms have meanings commonly used in the art unless otherwise specified; the definitions as provided herein are given with the purpose to facilitate understanding of certain terms used frequently herein and are not necessarily meant to limit the scope of the present disclosure; as used herein the term “about”, “c.a.”, “~”, and like, as used interchangeably herein, refers to the value and the range of ±10%; the terms “comprises”, “comprising”, “includes”, “including”, “having” and their conjugates mean “including but not limited to”, with this terms also encompassing the terms “consisting of” and “consisting essentially of”, which have their narrower meaning as known in the art, thus an embodiment described as comprising something also discloses embodiments consisting essentially of same and consisting exclusively of same; the singular forms “a”, “an”, and “the”, include plural referents unless the content clearly dictates otherwise; as used herein, a phrase in the form “A and/or B” means a selection from the group consisting of (A), (B) or (A and B); as used herein, a phrase in the form “at least one of A, B, and C” means a selection from the group consisting of (A), (B), (C), (A and B), (A and C), (B and C) or (A, and B, and C), and further combinations are envisaged for the lists comprising larger number of terms.

It is appreciated that, certain features of the invention, which are, for brevity, described in the context of separate embodiments, may also be provided in combination with other features in a single embodiment, unless technically infeasible. Conversely, features described in specific combinations of various features, which are, for clarity and demonstration, are described in the context of a single embodiment, may also be provided as separate embodiments individually or in any suitable sub-combination with other features and/or embodiments, as reasonable to the skilled artisan, suitable and operative. Certain features described in the context of various embodiments, including preferred features, are not to be considered essential features of those embodiments, unless the embodiment is inoperative without those elements.

### EXAMPLES

Reference is now made to the following examples, which together with the above descriptions illustrate some embodiments of the invention in a non-limiting fashion.

### Example 1

Preparing Extracts of Vernonanthura nudiflora, Baccharis Trimera, and Plantago major

The following extracting procedure was performed for each one of the indicated plants, separately. Wild-growing plants of each one of the plants Vernonanthura nudiflora, Baccharis trimera and Plantago major species were harvested and selected based on the overall conditions of the botanical material (also referred to herein as “biomass”), e.g., absence of parasites, absence of agrochemicals in the vicinity, etc. Next, botanical material of each one of the plants was carefully cleaned prior to extraction, leaving only the aerial parts (namely the plant parts residing above the soil, including the stems, leaves and petioles). The botanical material was washed with copious amount of water, and then was air-dried indoors, until a 50% reduction in its total mass was obtained, for about 7-8 days at room temperature or for about 3-4 days in a heated chamber (at 45° C.). The dried botanical material was then ground by using a mechanical mill to the final size of about 5.0-10.0 mm×1.0-2.0 mm×0.2-1.0 mm. The ground botanical biomass was then extracted during a period of 21 days in a reactor containing hydroalcoholic solution (at 70% ethanol in water solution, interchangeably termed herein a hydroethanolic solution), where the concentration of the botanical material in the hydroalcoholic solution was 0.2 g/ml. The solution was periodically agitated for about 1-2 hours, every 24-48 hours. The obtained extract was then filtered by gravity, through a filter with a pore size of 15 μm, and packaged in opaque and hermetically sealed containers, which were stored at room temperature in a cool and ventilated place, protected from exposure to light. To produce a mixture of three extracts, plant parts of the plant species Vernonanthura nudiflora, Baccharis trimera, and Plantago major were extracted individually, and the mixture thereof was prepared such in the mixing ratio 40% Vernonanthura nudiflora, 40% Baccharis trimera, and 20% Plantago major. The obtained extracts were stored at 4° C.

### Lyophilization of the Aqueous-Ethanolic Plant Extract

A dry product of the aqueous-ethanolic plant extract produced as detailed below was obtained by lyophilization as follows. A sample of plant extract (1 liter) in a water-ethanol phase was first subjected to removal of the ethanolic phase from the mother liquor. This step was performed by applying a vacuum of 750 mbar at 45° C. over a period of 80 hours. Upon completion of this drying step, the volume of the sample was reduced to 450 mL, therefore apparently all ethanol initially present was completely eliminated. A starch-based carrier (N-Zorbit 2144, Ingredion) was then added for homogenization of the final product by stirring for three (3) minutes, at a ratio of 1 gr modified starch for every 5 mL of the original extract (i.e., 200 g per liter of the original extract prior vacuum drying). The sample was then frozen at −80° C. for 72 hours (by introducing the sample in an ultra-freezer that reaches the mentioned temperature). Once the sample was frozen, it was lyophilized at −50° C. and 1 MPa vacuum, for 96 hours. A homogeneous and dry solid was obtained (234 gr). Once collected, the sample was stored at room temperature, preserving it from light.

### Example 2

Fractionation of Vernonanthura nudilora Hydroethanolic Extract by Organic Solvents

The hydroethanolic extract was further fractionated using different organic solvents, as detailed below.

First, a Vernonanthura nudilora hydroethanolic extract sample (100 ml) was air evaporated such that the concentration thereof was increased about 10-fold (i.e., to a final volume of about 10 ml) and contained mainly water as solvent, and then subjected to fractionation using the solvents hexane, ethyl acetate, chloroform, and butanol in the separating funnel, as known in the art. Briefly, extraction was performed independently with each one of the solvents, by adding the solvent (10 ml) to the concentrated V. nudilora hydroethanolic extract (7.5 ml), thoroughly shaking and separating into a solvent phase and a water phase. The above procedure was repeated (twice) to further extract any remaining agents from the water phase obtained. Next, each one of the extracts obtained was dried by evaporation (at 50° C.), and the dry extract was then dissolved in ethanol (0.2 ml of 70% ethanol).

Thus, sub-extracts of the original hydroethanolic extract were obtained.

### Example 3

This example describes a case report of treatment of a patient in need thereof by administering the composition according to Example 1. Briefly, the extracts of from Baccharis articulata, Plantago major, and Vernonanthura nudiflora, were mixed in proportion of 2:1:2, to obtain the mixed plant extract (also referred to herein as “MPE”).

A 17-year-old male presented for treatment of Asperger's syndrome (ICD-11, 6A02.0) and attention deficit hyperactivity disorder (ICD-11, 06A05). The patient has been diagnosed with the former at the age 4 and with the latter at the age 5. Prior to the visit the patient received non-pharmacological treatment only—special educational support and teaching aids at school. At the time of visit the patient was manifesting extreme anxiety, lack of focus, hyperactivity, and limited social interaction with either friends or parents.

Following obtaining the informed consent from the patient's legal guardian, they were instructed and agreed to use the provided medication according to instruction or to return the unused medication. The patient started receiving the MPE. The dose was 1/8 of a mL, administered per os as 1 mL of MPE diluted 1:8 in distilled water, four times a day.

After three weeks of therapy the patient demonstrated reduced hyperactivity and increased attention timespan. After 5 months of treatment the patient exhibited significantly reduced anxiety, improved focus on homework, enhanced social skills, and the hyperactivity was reduced to the level that the patient was reported to be able to play alone, seated, and quietly. After 8 months of treatment the patient maintained the positive improvements gained earlier. He has made notable progress in his school education, with significant improvements in managing his anxiety and enhancing his sociability.

According to treating professionals, such improvements are generally very rare, given the history of the patient, and are wholly unexpected without pharmacological intervention.

### Example 4

A 23-year-old female suffering from trisomy 21 (Down syndrome, ICD-LD40.0) was experiencing cognitive deficits and neuropathological features in line with Down-syndrome dementia (ICD-11, 6D85.9) following a vaccination against covid-19. The patient exhibited aggressive behavior, reduced focus, did not respond to basic requests, and there was a significant deterioration in basic tasks, including self-cleaning, and a decrease in communication skills. The patient was treated with sedatives and vitamin C without any improvement for several months. The prognosis indicated that this regression could be permanent.

Following obtaining the informed consent from the patient's legal guardian, they were instructed and agreed to use the provided medication according to instruction or to return the unused medication, the patient started MPE treatment 6 months after diagnosis, along with light sedatives and Vitamin C. The dosage followed was 10 ml of diluted 1:8 (in distilled water) MPE per os 2 times a day.

After 15 days of treatment with MPE, the patient began to show improvement in communication skills and behavior. After 3 months of treatment with MPE, improvements continued, with the patient returning to the state before the onset of cognitive deficit symptoms.

At this point the treatment with MPE has been stopped, but resumed after 2 months because of the regression and loss of progress the patient had previously made. Upon resuming of the MPE treatment, a noticeable improvement again was observed. The patient experienced significant improvement in aggressive behavior, focus, and overall verbal and non-verbal communication skills and returned to the state before the vaccination-induced deterioration.

According to treating professionals, such improvements are generally very rare, and are wholly unexpected since this kind of deterioration in trisomy-21 patients is usually permanent.

### Example 5

A 29-year-old male suffering from an autism-spectrum disorder (ICD-11, 6A02) accompanied with a general anxiety (ICD-11, 6B00) presented for treatment. The patient has been diagnosed at the age of 5, and has been treated with the combination of olanzapine, lorazepam, brexpiprazole, citalopram, and alprazolam, during the course of the preceding 7 years, with variable outcomes. The patient has been manifesting very poor concentration with no focus, very poor language skills, reduced to almost no speaking, aggressive behavior.

Following obtaining the informed consent from the patient's legal guardian, they were instructed and agreed to use the provided medication according to instruction or to return the unused medication. The patient started receiving the MPE as adjunctive to his normal therapy. The dose was 2 ml of diluted 1:8 MPE (in distilled water), per os 3 times a day.

After one month of the combined therapy, the patient showed notable improvement in focus and behavior. After 5 months of treatment with MPE and the previous medications, the patient maintained and increased improvement in behavior, was able to use electronic devices without frustration, maintained focus on his tasks, watched TV while sitting down without anxiety. Communication skills also improved significantly leading him to communicate his needs and feelings. Thereafter the medications were gradually tapered off and eventually completely stopped. The patient is currently prescribed citalopram on an as-needed basis.

At the last visit the patient showed sustained and progressive improvement. The patient engages in shopping independently, effectively uses electronic devices, demonstrates increased attention span. Communication skills have notably advanced; he now speaks to express his needs and desires with clarity. Behaviorally, there has been a significant reduction in aggressiveness, and he is observed to be more adaptive in his daily functioning. The patient also shows enhanced problem-solving abilities, as seen in his capacity to resolve simple games.

According to treating professionals, such improvements are generally very rare, and are particularly unexpected since the medications from his regular treatment have been stopped.

### Example 6

A 18-year-old male suffering from a continuous symptomatic schizophrenia (ICD-11, 6A20.2) presented for treatment. The patient has been suffering for the preceding seven years, and was treated with risperidone, clonazepam, and aripiprazole, all discontinued due to worsening of the symptoms. Upon presentation the patient was symptomatic, experienced significant auditory, olfactory, gustatory, and tactile hallucinations, along with sleeping problems. The patient experienced urges to comply with the instructions received by auditory hallucinations.

Following obtaining the informed consent from the patient's legal guardian, they were instructed and agreed to use the provided medication according to instruction or to return the unused medication. The patient started receiving the MPE as adjunctive to his normal therapy. The dose was 15 ml of diluted 1:8 MPE (in distilled water), per os, 4 times a day. The accompanying treatment was hypnotics to mitigate sleep disturbances.

One month after the commencement of the treatment, the patient observed noticeable improvements, including a reduced frequency and intensity of hallucinations and enhanced sleep quality, and after five months of treatment the hypnotics were withdrawn. The patient reported at this point complete resolution of auditory and visual hallucinations. On the later follow-up patient presented regained mental stability and discontinued all medications, following a comprehensive remission of symptoms. The absence of visual and auditory hallucinations has enabled the patient to resume educational pursuits, entrepreneurial activities, and a consistent exercise regimen.

According to treating professionals, such improvements are particularly unexpected given the sustained effect of the treatment and the failure of prior pharmacotherapy.

### Example 7

A 5-year-old male suffering from trisomy 21 (Down syndrome, ICD-11, LD40.0) was experiencing cognitive deficits and developmental retardation presented for treatment. The patient exhibited low control of basic tasks, including self-cleaning, used diapers, exhibited behavior problems at school, showed a lack of social or communication skills and reasoning abilities. Prior to visit, the patient attended special schools, received support from special educators, and did not receive pharmacotherapy.

Following obtaining the informed consent from the patient's legal guardian, they were instructed and agreed to use the provided medication according to instruction or to return the unused medication. The patient started receiving the MPE as adjunctive to his normal therapy. The dose was 3 ml of diluted 1:8 MPE (in distilled water), per os, 4 times a day.

After 2 months of the treatment the patient showed improvement in school performance and behavior. The patient was able to stop using diapers and demonstrated improved reasoning and communication skills. Presented after 2 years of use the patient has shown further improvement in reasoning skills and is communicating more effectively with family and others, becoming more sociable.

According to treating professionals, such improvements are particularly remarkable and unexpected given patient's condition and MPE as monotherapy.

### Example 8

A 29-year-old female suffering from epilepsy with myoclonic-atonic seizures (ICD-11, 8A61.2Y) and alternating hemiplegia (ICD-11, MB53.0) was complaining on symptoms upon presentation. The patient has been previously treated with levetiracetam 500 mg b.i.d., and received occupational therapy and physiotherapy.

Following obtaining the informed consent, the patient was instructed and agreed to use the provided medication according to instruction or to return the unused medication. The patient started receiving the MPE as adjunctive to his normal therapy. The dose was 5 ml of diluted 1:8 MPE (in distilled water), per os, 2 times a day. The patient continued non-pharmacological interventions and levetiracetam, maintaining a two-hour interval between the administrations.

After 2 months of treatment no episodes of rigidity were reported and atonic seizures reduced frequency. After 5 months of treatment the patient maintained the reduced frequency (few episodes per month) of tonic and atonic seizures, with reemerging rigidity by a few episodes per month.

According to treating professionals, such improvements are unexpected with no pharmacotherapy modifications and may be attributed to the MPE intervention.

1.-25. (canceled)

26. A method of treating a neurological and/or psychiatric disorder in a subject in need thereof, comprising administering to said subject a composition comprising a hydroethanolic plant extract or any fraction thereof, wherein said plant is selected from Vernonanthura nudiflora, Plantago major and a Baccharis family species selected from Baccharis crispa, Baccharis trimera, and/or Baccharis articulata and any combination thereof.

27. The method according to claim 26, wherein said extract is obtainable, preferably essentially identical to an extract obtained by, a process comprising combining the biomass of at least one of said plant(s) with a hydroethanolic mixture comprising 20 to 50 volume percent of water, and extracting said biomass into said hydroethanolic mixture at a temperature between about 15° C. and 40° C., preferably between 20° C. and 25° C., for a time interval of between about 3 and 35 days, preferably between about 18 and 23 days, further preferably wherein the concentration of said biomass being extracted is between about 13% and 33% weight by volume of the total extraction mixture.

28. The method according to claim 26, wherein said plant is a combination of Vernonanthura nudiflora, Plantago major, and a Baccharis family species selected from Baccharis crispa, Baccharis trimera, and/or Baccharis articulata.

29. The method according to claim 26, wherein a weight ratio of each of the plants is between 20 and 50% wt, preferably, the ratio between the plants is 2:2:1 with Plantago major being the minor component.

30. A method according to claim 26, said extract containing less than 5-20% by weight of the compounds listed in Table 1, or is essentially devoid of these compounds.

31. The method according to claim 26, wherein said neurological and/or psychiatric disorder is a disease or disorder classified in a subclass 06 or 08 of the 11^{th} revision of World Health Organization International Classification of Diseases.

32. The method according to claim 26, wherein said neurological and/or psychiatric disorder is a neurodevelopmental disorder, optionally selected from an autism spectrum disorder, an attention deficit hyperactivity disorder, or Down Syndrome-associated development retardation, and further optionally wherein said autism spectrum disorder Asperger's syndrome.

33. The method according to claim 26, wherein said neurological and/or psychiatric disorder is an anxiety or fear-related disorder.

34. The according to claim 26, wherein said neurological and/or psychiatric disorder is a primary psychotic disorder, optionally schizophrenia.

35. The method according to claim 26, wherein said neurological and/or psychiatric disorder is a seizures disorder, optionally selected from an epilepsy, or a hemiplegia.

36. The method according to claim 26, wherein said hydroethanolic plant extract is a dried hydroethanolic plant extract.

37. The method according to claim 26, wherein said extract is a liquid hydroethanolic plant extract.

38. The method according to claim 26, wherein said method comprises administering to a subject in need thereof a therapeutically effective amount of said composition.

39. The method according to claim 38, wherein said administering is by a route selected from the group consisting of intravenous administration, oral administration, intramuscular administration, and intraperitoneal administration.

40. The method according to claim 38, wherein said administering comprises providing to said patient a daily amount of plant extract equivalent to a dose between 10 mg and 300 mg, on dried basis, divided into doses according to a suitable regimen.

41. The method according to claim 40, wherein said dose is between 10 mg and 120 mg on dry basis, divided into doses according to a suitable regimen, preferably wherein said neurological and/or psychiatric disorder is a neurodevelopmental disorder.

42. The method according to claim 40, wherein said dose is between 100 mg and 300 mg on dry basis, preferably wherein said neurological and/or psychiatric disorder is a primary psychotic disorder.

43. The method according to claim 40, wherein said regimen is selected from once daily, twice daily, three times daily, and four times daily.

44. The method according to claim 26, wherein said method further comprises administering to said subject an additional therapeutic agent.

45. The method according to claim 26, wherein said composition is in a form selected from the group consisting of a tincture, a syrup, an elixir, a capsule, and a tablet.
